(19) United States
(12) Patent Application Publication
(10) Pub. No.: US 2011/0086048 A1
(43) Pub. Date: Apr. 14, 2011

(54) DETECTION OF HUMAN IMMUNODEFICIENCY VIRUS CO-RECEPTOR TROPISM IN AVIREMIC SUBJECTS

(75) Inventor: Mario Stevenson, Shrewsbury, MA (US)

(73) Assignee: UNIVERSITY OF MASSACHUSETTS, Shrewsbury, MA (US)

(21) Appl. No.: 12/866,424

(22) PCT Filed: Feb. 6, 2009
(86) PCT No.: PCT/US09/33313

§ 371 Date: Dec. 10, 2010

Related U.S. Application Data
(60) Provisional application No. 61/026,623, filed on Feb. 6, 2008.

(51) Int. Cl. A61K 39/395; C12Q 1/68; A61K 38/18; A61P 31/18

(52) U.S. Cl. 424/173.1; 435/6; 424/130.1; 424/172.1; 514/3.8

(57) ABSTRACT

Methods for detecting human immunodeficiency virus (HIV) co-receptor tropism or replication-competent virus in aviremic subjects, and methods of selecting optimal therapies for aviremic subjects.

### CLAIM OF PRIORITY

This application claims the benefit of U.S. Provisional Patent Application Ser. No. 61/026,623, filed on Feb. 6, 2008, the entire contents of which are hereby incorporated by reference.

### FIELD OF THE INVENTION

The invention relates to human immunodeficiency virus (HIV) co-receptor tropism detection assays, and methods of selecting optimal therapies for aviremic subjects.

### BACKGROUND OF THE INVENTION

HIV-1 positive individuals undergoing combination antiviral therapy (i.e., receiving two or more anti-HIV-1 compounds) can exhibit decreased viral loads in the peripheral blood. In some eases, after several weeks or months of therapy, HIV-1 RNA cannot be detected in the peripheral blood using standard methods. Unfortunately, if such aviremic patients stop therapy, the HIV can rebound very rapidly, indicating the presence a reservoir of replication-competent virus in the patient that is not detectable using standard methods. It would be desirable to select a therapy that is specifically targeted to the genotype of that viral reservoir, however, the limited sensitivity of standard HIV tropism detection assays provides a challenge to further advances in therapy.

### SUMMARY OF THE INVENTION

The invention is based on the discovery that patients having no virus detectable in the blood by known means, e.g., patients undergoing drug therapy. Rich as combination drug therapy, can nevertheless harbor replication-competent HIV, and that this viral reservoir is indicated by the presence of 2-LTR circles in peripheral blood mononuclear cells (PBMC's). The presence of the 2-LTR circles indicates the persistence of viral spread in those patients, in spite of the fact that these patients have no detectable virus in the blood. Since the 2-LTR circles are related to the replication-competent virus, the genotype of the circles can be used to determine the tropism of the virus. This information can be used to guide the selection of antiviral medications that inhibit the entry of virus into cells (“entry inhibitors”) by blocking the virus/cell surface receptor interaction, for subjects who are aviremic (i.e., have no detectable levels of cell-free HIV viral RNA in their blood).

Thus, the invention provides methods for determining co-receptor tropism of replication competent virus in an HIV-positive subject, e.g., a mammal, e.g., a primate, e.g., a human, who has less than 50 cell-free viral RNA molecules/ml of serum, in general, the methods include providing a sample comprising a cell (e.g., a peripheral blood in cell (PBMC)) from the subject; detecting an HIV 2-LTR circle DNA molecule in the sample; and determining the co-receptor tropism of the 2-LTR circle DNA. As the 2-LTR circles tire derived from infectious virus, the co-receptor tropism of 2-LTR circle DNA indicates the co-receptor tropism of the replication competent virus in the subject.

In some embodiments, determining the co-receptor tropism of the 2-LTR circle comprises determining the genotype of (e.g., sequencing) the DNA. In some embodiments, the DNA molecule is amplified before determining the genotype of the DNA, e.g., using polymerase chain reaction (PCR) with primers specific for an envelope protein or a portion thereof, e.g., the V3 loop. In some embodiments, the envelope protein is gp120 or gp41.

In some embodiments, determining the co-receptor tropism of the 2LTR circle comprises performing a phenotypic tropism assay, e.g., a cell-based assay as desert bed herein.

In some embodiments, the subject is being treated with highly active antiretroviral therapy (HAART). In some embodiments, cell-free HIV viral RNA cannot be detected in the blood of the mammal.

In some embodiments, the co-receptor tropism of the 2LTR circle DNA indicates that the replication competent virus is primarily M-tropic, primarily T-tropic, primarily dual-tropic; or (q mixed tropism.

in some embodiments, the methods further include selecting an entry inhibitor based on the co-receptor tropism of the replication competent virus. For example, the methods can include selecting a CCR5-specific entry inhibitor based on the presence of co-receptor tropism for CCR5 or of dual tropism, or selecting a CXCR4-specific entry inhibitor based on the presence of co-receptor tropism for CXCR4.

Also provided herein are methods for treating an HIV-infected subject, e.g., a mammal, e.g., a primate, e.g., a human, who has less than 50 cell-free viral RNA molecules/ml of serum. The methods generally include providing a sample comprising a cell from the subject; detecting an HIV 2-LTR circle DNA molecule in the sample; determining the co-receptor tropism of the 2-LTR circle DNA; determining co-receptor tropism of replication-competent virus in the subject based on the co-receptor tropism of the 2-LTR circle DNA; selecting an entry inhibitor suitable for the HIV co-receptor tropism of the virus in the subject; and administering an effective amount of the selected entry inhibitor to the subject, thereby treating the subject.

In some embodiments, the methods include selecting a CCR5-specific entry inhibitor for a subject in whom the co-receptor tropism is for a receptor other than CXCR4. In some embodiments, the methods include selecting a CXCR4-specific entry inhibitor for a subject in whom the co-receptor tropism is for CXCR4.

In some embodiments, the methods described herein include a step of confirming that the subject has less than about 50 cell-free viral RNA molecules/ml of serum.

An HIV-positive individual is one who produces antibodies that specifically bind to an HIV viral protein.

Cell-free HIV viral RNA is RNA that is not associated with a cell (e.g., RNA in plasma).

Analysis of 2-LTR circles can be applied to determine HIV genotype(s) in infected individuals. Genotype analysis is useful for determining therapy. For example, therapy with CCR5 inhibitors is contraindicated in patients infected with a CXCR4-tropic virus because CCR5 inhibitors are ineffective against CXCR4-tropic viruses, and may promote outgrowth of pathogenic strains. (CXCR4 refers to Chemokine, CXC motif, Receptor 4; CCR5 refers to Chemokine, CC motif, Receptor 5). Accordingly, the invention includes methods in which the genotype of 2-LTR, circle DNA from a subject e.g. an aviremic subject) is determined. In some embodiments, the genotyping includes determining the co-receptor tropism of the HIV virus (e.g., CCR5 or CXCR4 tropism, e.g., by evaluating the envelope gene). In some embodiments, therapy with a CCR5 inhibitor is indicated for a subject infected with a virus that is not CXCR4-tropic. In some embodiments, therapy with a CCR5 inhibitor is not indicated for a subject infected with a CXCR4-tropic virus. Likewise, in some embodiments, therapy with a CXCR4 inhibitor is indicated for a subject infected with a CXCR4-tropic virus. In some embodiments, therapy with a CXCR4 inhibitor is contraindicated for a subject infected with a CCR5-tropic virus.

Unless otherwise defined, all technical and scientific terms used herein have the same meaning as commonly understood by one of ordinary skill in the art to which this invention belongs. Although suitable methods and materials for the practice or testing of the present invention are described below, other methods anti materials similar or equivalent to those described herein, which are well known in the art, can also be used. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent front the following detailed description, and from the claims.

### BRIEF DESCRIPTION OF THE DRAWINGS

FIGS. 1A and 1B are graphs of viral DNA (copies/10⁵ cells) versus time post RT inhibition, showing data for HIV-1_{LAI} and HIV-1_{ADA}, respectively.

FIGS. 2A-2D are graphs of HIV-1 RNA or genomes versus time in weeks, showing the data for patients Gu, Sm, Za, and Ha, respectively.

FIG. 3 is a data point plot of number of virus-positive and virus-negative cultures for patient designations.

### DETAILED DESCRIPTION

invention relates to a methods of determining the co-receptor tropism of replication-competent HIV in aviremic HIV-positive patients, for example, patients undergoing antiviral drug therapy, by detecting and genotyping HIV 2LTR circles in the patients cells. Present methods of determining co-receptor tropism rely on the presence of plasma viral RNA at least 100 to several thousand molecules of viral RNA/ml plasma), however, as discussed herein, patients without detectable plasma viral RNA may still retain replication-competent virus. The present methods can be used to determine co-receptor tropism of that replication-competent virus in patients who are aviremic, i.e., who test negative for plasma viral RNA (below 40 or 50 molecules of viral RNA/ml plasma). This allows the selection and administration of entry inhibitors that are specific for the co-receptors used by the virus present in the subject.

### Viral Tropism

HIV entry into cells is mediated through sequential interactions between HIV envelope glycoproteins (Env) and two cellular molecules: CD4 and a co-receptor, typically either CCR5 or CXCR4 (Coakley et al., Curr Opin Infect Dis. 2005 February; 18(1):9-15). The envelope glycoprotein of HIV-1 consists of a complex of gp120 and gp41. gp120 determines viral tropism (the specificity of a virus for a particular cell or tissue) by binding to a specific co-receptor, while gp41 mediates fusion between viral and cellular membranes. See, e.g., Moore and Doms, Proc Natl Acad. Se; USA. 2003; 100:10598-10602.

Macrophage (M-tropic, or R5) strains of HIV-1 use the β-chemokine receptor CCR5 for entry. The CCR5 co-receptor is used by almost all primary HIV-1 isolates regardless of viral genetic subtype. T-tropic (or X4) isolates use the α-chemokine receptor. CXCR4, for entry. Dual-tropic (D) HIV-1 strains are able to use both CCR5 and CXCR4 as co-receptors for viral entry, and may be transitional strains of the HIV-1 virus. Mixed-tropic (M) virus populations may contain various combinations of R5 virus, X4 virus, and/or dual-tropic viruses. See, e.g., (Coakley et al., Curr Opin Infect Dis, 2005 February; 18(1):9-15: Deng, et al., Nature 1990; 381(6584):661-6; Feng et al., Science 1990; 272 (5263):872-7).

Present methods of determining viral tropism use plasma viral RNA, and thus cannot be used on subjects who are aviremic.

### Sample Preparation

A variety of biological samples can be analyzed by the methods of the invention, including blood and solid-tissue biopsies (e.g. a lymph node biopsy). For example, blood can be collected from an HIV-positive individual undergoing combination therapy. PBMC can be isolated by standard ficoll-based isolation procedures. The PBMC are then lysed and the total or extrachromosomal DNA isolated.

Total cellular DNA can be extracted by lysing the PBMC in detergent, digesting the cellular protein, and precipitating the DNA (Pauza et al., Virology, 205:470-478, 1984; and Panther et al., J. Acquir. Immune. Defic. Syndr. Hum. Retro., 17:303-313; 1998). Extrachromosomal DNA can be isolated by any method known in the art, including standard alkaline lysis, Hirt extraction, or guanidinium thiocyanate precipitation (Jurrians et al., J. Gen. Virol., 73:1537-1541, 1992; Stevenson et al., J. Virol, 64:2421-2425, 1990; and Sambrook et al., eds., Molecular Cloning: A Laboratory Manual. 2nd, ed., Cold Spring Harbor Laboratory, Cold Spring Harbor Laboratory Press, Cold Spring Harbor, N.Y., 1989).

Although the standard alkaline lysis technique is best known for isolating plasmid DNA from bacteria, this technique can also be used according to the invention to isolate 2-LTR circles from mammalian cells. The Spin Miniprep Kit available from Qiagen (Cat. No. 27104) is especially useful for this purpose. The methods of the invention include the use of this technique to isolate and purify 2-LTR circle DNA.

When possible, extrachromosomal DNA, instead of total DNA, should be isolated since the another of target 2-LTR circles per microgram of extrachromosomal DNA is expected to be tar greater than the number of 2-LTR circles per microgram of total cellular DNA.

### 2-LTR Circles and Methods of Detection

In vitro studies of retroviruses have shown that the first, evidence of reverse transcription is unintegrated viral DNA appearing in the cytoplasm, which is transported to the nucleus within hours after infection of a cell (Shank et al. J. Virol. 25:104-114, 1978; Clayman et al., Science; 206:582-584, 1979; and Stevenson et al., EMBO J., 9:1551-1560, 1990). In the case of HIV-1, this unintegrated DNA exists in several forms, including incompletely or completely reverse-transcribed linear DNA, circular DNA containing one LTR, and circular DNA containing two LTRs (2-LTR circles). 2-LTR circles are identical to integrated proviruses, except that the ends of the LTR are joined in head-to-tail fashion via a covalent linkage.

PCR can be used to specifically amplify a small segment to few hundred base pairs) spanning the 2-LTR junction. The PCR is specific for 2-LTR circles, since no proviruses, single LTR circles, or other incomplete viral reverse transcription products will be amplified. Methods of detecting and/or quantifying 2-LTR circles are described herein and in the art. See, e.g., U.S. Pat. Nos. 7,232,657 and 6,797,464; and U.S. Pat. Pub. No. 2005-0064393.

For example, 24-LTR circles can be detected using known techniques, including those that do not require nucleic acid amplification, such as Southern blotting. The DNA sample obtained as described herein can be hybridized with 2-LTR circle-specific probes that are directly or indirectly labeled with chromogenic, radioactive, fluorescent, or luminescent labels.

Where amplification of the 2-LTR circles is dewed, e.g., before a detection step, the 2-LTR circles can be amplified by any method well known in the art. These methods include polymerase chain reaction (PCR: U.S. Pat. Nos. 4,683,195 and 4,683,202) and variants thereof. Another suitable nucleic acid amplification method ms ligation chain reaction (LCR) or variants thereof (Landegran et al., Science, 241:1077-1080, 1988; and Nakazawa et al., Proc. Natl. Acad. Sci. USA, 91:360-364, 1994). Methods for performing quantitative PCR using 2LTR-specific primers is described in Stevenson et al., J. Virol. 4:2421-2425 (1990).

Other amplification methods include: self-sustained sequence replication (Guatelli et al., Proc. Natl. Acad. Sci. USA, 87:1874-1878, 1990), transcriptional amplification system (Kwoh, et al., Proc. Natl. Acad. Sci. USA, 86:1173-1177, 1989), and Q-Beta Replicase (Lizardi et al., Bio/Technology, 6:1197, 1988).

However the 2-LTR circles are detected, a threshold level of 2-LTR circles per million cells is useful to define meaningful numbers of the circles. If the assay is capable of single-molecule sensitivity, a base threshold can be established at one circle per million PBMC. This threshold is appropriate when determining whether eradication of HIV has been achieved in a patient. Whenever a patient tests above this threshold, the patient is said to exhibit active viral infection. Whenever a patient tests below the threshold, the patient is said to have undetectable levels of infection and may be a candidate for removal from antiviral therapy. In other contexts, such as when the level of 2-LTR, circles is used to determine the efficacy of any antiviral regime, thresholds above one per million PBMC can be appropriate (e.g., 10, 50, 100, or 250 circles/10⁶ PBMC).

Any of the above methods can be combined in a method of the invention to achieve suitable detection efficiencies. The amplification products can then be genotyped using methods known in the art to determine the predominant tropism of the replication-competent virus.

Detection of HIV viral RNA

Various assays have been developed to detect HIV viral RNA in plasma. A common HIV-1 detection assay utilizes quantitative polymerase chain reaction (PCR) as a means to amplify and detect viral RNA present in patient plasma. For example, plasma viral RNA in a sample can be measured using the AMPLICOR® HIV Monitor Test kit (Roche Molecular Systems, Inc., Branchburg, N.J.), employing HIV-1-specific quantitative PCR, following manufacturer's directions. The threshold of detection for this standard HIV-1 RNA detection assay is about 40-50 viral RNA molecules per milliliter of plasma. In some embodiments, the assay is an “ultrasensitive” assay, which allows input of RNA from 10-fold more plasma per amplification reaction, and has a detection threshold of about 4-5 molecules/ml plasma. See, e.g., Sun et al., J. Clin. Microbiol. 1998 Oct.; 36(10): 2964-2969.

### Determining Co-Receptor Tropism Based on 2-LTR Circles.

Viral tropism plays a role in HIV pathogenesis. Knowledge of the HIV tropism in an individual is useful for monitoring the course of infection and determining appropriate therapy (e.g., by selecting therapy with antagonists directed against the receptor tropism of the HIV strain in the infected individual, and by avoiding therapy with antagonists that would be ineffective and/or promote outgrowth of pathogenic strains in the individual).

Methods of determining co-receptor tropism are known in the art and include phenotypic assays cell-based assays) and genotyping (e.g., using nucleic acid sequencing or heteroduplex tracking assays). Some methods are described, e.g., in Poveda et al., J. Med. Virol., 79(8):1040-1046, 2007; Foeglein and Walter, Eur. Med. Res., 12(9):473-482; 2007; and Van Baelaen et al., J. Virol. Meth., 146:61-73, 2007.

Genotyping generally includes predicting, viral tropism based on the sequence of a viral protein, e.g., an envelope protein, i.e., gp120 or gp41. Most of the genetic determinants of coreceptor usage reside in the HIV envelope, in particular, the V3 loop (Fouchier et al., 1992; J. Virol. 66:3183-3187; Hoffman et al., 2002; J. Virol. 76:3852-3864; Jensen et al., J Virol. 2003 December; 77(24):13376-88). Thus, this region can be used for genotypic predictors of coreceptor usage. On approach, known as the “11/25 rule” classifies a virus as X4 if positively charged amino acids (lysine or arginine) are present at positions 11 and/or 25 of the V3 loop (DeJong, et al., 1992; J. Virol. 66:6777-6780; Fouchier et al., 1995; J. Clin. Microbiol. 33:906-911; Fouchier et al., 1992; J. Virol. 66; 3183-3187). Boinformatics-based genotypic predictors, such as neural networks (Resch et al., 2001; Virology 288:51-62), position-specific; scoring matrices (Jensen et al., 2003; AIDS Rev. 5:104-112), and support vector machines (SVMs), indicate that the coreceptor phenotypes may be accurately determined with genotypic predictors (Sing et al., abstr. 30. Abstr. 1st Int. Workshop Targeting HIV Entry, Bethesda, Md.) when tested on clonally derived sequence data. See, e.g., Garrido et al. J Clin Microbiol. 2008 March; 46(3):887-91. Epub 2008 Jan. 16; Skrabal et al., J Clin Microbiol. 2007 February; 45(2):279-84, Epub 2006 Nov. 22; and Raymond et al., AIDS. 2008 Sep. 12; (14):F11-6; Low et al., AIDS Rev. 2008 July-September; 10(3); 143-51: and Sierra et al. Genotypic Coreceptor Analysis. 2007; Eur J. Med Res 12:453-462.

In addition, there are several commercially available tropism assays: cell-based phenotypic recombinant virus assays (RVAs) (e.g., single-cycle assays as described in Whitcomb et al., Antimicrob Agents Chemother, 2007; 51:566-575; in Poveda et al., AIDS: Volume 2007; 21(11):1487-1490; and in Dam et al., 5th International Workshop on HIV Drug Resistance and Treatment Strategies, Jun. 4-8, 2001; Scottsdale, Ariz.; and embodied in the TRT assay, Monogram Diagnostics' TROFILE™ assay, and PHENOSCRIPT™ assay from VIRalliance); and a heteroduplex tracking assay HTA (see, e.g., WO2007084568, and Pathway Diagnostics' SENSITROP™ and SENSITROP II™ (available through Quest Diagnostics, Inc.)). Exemplary HTA assay methods include amplification of the V3 env region from the 2-LTR circles; hybridization of the patient HIV V3-DNA with standard HIV V3-DNA probes from CCR5-tropic HIV strains; and electrophoretic separation and detection of CXCR4/CCR5 heteroduplexes in non-denaturing polyacrylamide gels. Combinations of phenotypic and genotypic assays, e.g., the XTRACKC/PHENX-R™ assay (from in Pheno) and an assay from Virco. For additional information on assays for determining co-receptor tropism, see, e.g., Braun and Wiesman, Eur J Med Res. 2007 Oct. 15; 12(9):463-72; Skrabal et al., J Clin Microbiol. 2007 February; 45(2):279-84. Epub 2006 Nov. 22; WO967429; WO2007088201; WO0179540; WO0157245; and U.S. Pat. Nos. 6,057,102 and 6,727,060. One of skill in the art would readily be able to adapt the art-known methods for use with 2-LTR DNA in place of.

### Antiviral Drugs

A number of HAART regimens are presently used; exemplary regimes can include combinations or Nucleoside Reverse Transcriptase Inhibitors (NRTIs), Protease Inhibitors (PI), and/or non-nucleoside reverse transcriptase inhibitors (NNRTIs). In some embodiments, the subject is being treated with a regimen that includes 2 NRTIs plus an NNRTI and/or a PI. In some embodiments, the subject's treatment includes an additional drug or drugs that targets viral integration into genomic DNA (i.e., an integration inhibitor, e.g., raltegravir). The present methods can include the intensification of the subject's treatment by the addition of art additional drug or drugs that inhibits entry of the virus into cells (entry inhibitors).

NRTIs

NRTIs are the nucleoside and nucleotide analogs, which replace the normal endogenous nucleotides/nucleosides, preventing the reverse transcriptase from transcribing viral RNA. Exemplary NRTIs are listed in Table A.

TABLE A
Nucleoside Reverse Transcriptase Inhibitors (NRTIs)

| | | Pharmaceutical |
|---|---|---|
| Brand Name | Generic Name | Company |
| COMBIVIR | zidovudine + lamivudine | GlaxoSmithKline |
| EMTRIVA | emtricitabine | Gilead Sciences |
| EPIVIR | lamivudine | GlaxoSmithKline |
| EPZICOM | abacavir + lamivudine | GlaxoSmithKline |
| RETROVIR | zidovudine | GlaxoSmithKline |
| TRIZIVIR | abacavir + zidovudine + | GlaxoSmithKline |
| | lamivudine | |
| TRUVADA | tenofovir DF + | Gilead Sciences |
| | emtricitabine | |
| VIDEX & VIDEX EC | didanosine | Bristol-Myers Squibb |
| VIREAD | tenofovir disoproxil | Gilead Sciences |
| | fumarate (DF) | |
| ZERIT | stavudine | Bristol-Myers Squibb |
| ZIAGEN | abacavir | GlaxoSmithKline |
| RACIVIR | | Pharmasset |
| | amdoxovir | RFS Pharma |
| | apricitabine | Avexa Limited |
| | elvacitabine | Achillion |
| | | Pharmaceuticals |

PIs

PIs inhibit the activity of the HIV protease, preventing the production of functional viral particles. Exemplary PIs are listed in Table B.

TABLE B
Protease Inhibitors (PIs)

| Brand Name | Generic Name | Pharmaceutical Company |
|---|---|---|
| AGENERASE | amprenavir | GlaxoSmithKline and Vertex |
| APTIVUS | tipranavir | Boehringer Ingelheim |
| CRIXIVAN | indinavir | Merck & Co |
| INVIRASE | saquinavir | Hoffmann-La Roche |
| KALETRA | lopinavir + ritonavir | Abbott Laboratories |
| LEXIVA | fosamprenavir | GlaxoSmithKline |
| NORVIR | ritonavir | Abbott Laboratories |
| PREZISTA | darunavir | Tibotec |
| REYATAZ | atazanavir | Bristol-Myers Squibb |
| VIRACEPT | nelfinavir | Pfizer |

NNRTIs

NNRTIs bind to reverse transcriptases and prevent the transcription of viral RNA. Exemplary NNRTIs are listed in Table C.

TABLE C
NON-Nucleoside Reverse Transcriptase Inhibitors (NNRTIs)

| Brand Name | Generic Name | Pharmaceutical Company |
|---|---|---|
| INTELENCE | etravirine | Tibotec |
| RESCRIPTOR | delavirdine | Pfizer |
| SUSTIVA | efavirenz | Bristol-Myers Squibb |
| VIRAMUNE | nevirapine | Boehringer Ingelheim |
| | rilpivirine | Tibotec |

Entry Inhibitors

Entry inhibitors (also known as fusion inhibitors) target the gp120 or gp41 HIV envelope proteins, or the CD4 protein, or CCR5 or CXCR4 receptors on a CD4 cell's surface. A number of entry inhibitors are known in the art, e.g., enfuvirtide (Trimeris and Hoffmann-La Roche, targets gp41); maraviroc (SELZENTRY™, Pfizer, targets CCR5; see, e.g., Mayer et al. AIDS 2006—international AIDS Conference; Aug. 13-18, 2006, Toronto, Canada. Abstract THLB0215); vicriviroc (Schering-Plough Corporation, targets CCR5); PRO 140 (progenies Pharmaceuticals, targets the CD4 protein); Pentafuside (T-20, Hoffman-LaRoche/Trimeris, targets gp41, see Kilby et al., Nat. Med, 1302-1307, 1998); T-1249 (Hoffman-LaRoche/Trimeris, targets gp41); and NBD-556 and analogs thereof, e.g., as described in Madani et al., Structure 2008 November; 16:1689-1701. Additional entry inhibitors include the CXCR4 antagonists AMD 3100 (Johnson Matthey/Anormed Pharmaceuticals). T22 (Seikagaku Pharmaceuticals), and ALX40C-4C (Allelix Pharmaceuticals), and the natural CCR5 ligands (e.g. MIP-1α, MIL-1β, and RANTES), and analogs thereof (e.g., TAK-779 and AOP-RANTES). See, e.g., Eron, PRN Notebook, 5(3):10-14 (2000).

Inhibitory antibodies targeting the various proteins, e.g., antibodies that specifically bind gp120, gp41, CD4, CCR5, or CXCR4, can also be used e.g., TNX-355 (Tanox, Inc., targets the CD4 protein).

Dosages, specific formulations, and routes of administration of HIV antiviral drugs are known in the art. See, e.g., Physician's Desk Reference, 63rd edition (Medical Economies Company, Montvale, N.J., 2009); Panel on Antiretroviral Guidelines for Adult and Adolescents. “Guidelines for the use of antiretroviral agents in HIV-1-infected adults and adolescents.” Department of Health and Human Services. Nov. 3, 2008; pp 1-139 (available at aidsinfo.nih.gov/ContentFiles/AdultandAdoleacentGL.pdf); and Kuritzkes et al. (1999. AIDS 13:685-694).

### EXAMPLES

The invention will be further described in the following example, which does not limit the scope of the invention described in the claims.

### Example 1

Blood samples were obtained using standard techniques from 20 HIV-1-infected individuals who began and continued to receive combination anti-HIV drug therapy. All of these patients exhibited a period of time in which, after commencement of combination therapy, no plasma viral RNA could be detected by quantitative PCR. PBMC were isolated from each blood sample, and the extrachromosomal DNA was purified using the Spin Miniprep Kit available from Qiagen as Cat. No. 27104, generally following the manufacture's directions.

HIV-1 2-LTR circles were detected by quantitative PCR using the 2-LTR-specific primers described in Stevenson et al., J. Virol., 64:2421-2425 (1990). The (−) strand primer spanned nucleotides 9591 to 9610 (or 507-526) of the HXB2 strain of HIV-1, while the (+) strand primer spanned nucleotides 9650-9669 (or 566-585) of the HXB2 strain of HIV-1 (Ratner et al., Nature, 313:277-284, 1985). Plasma viral RNA in each sample was also measured using the Amplicor HIV Monitor□ kit (Roche Molecular Systems, Inc., Branchburg, N.J.), employing HIV-1-specific quantitative PCR, following manufacturer's directions. The threshold of detection for this standard HIV-1 RNA detection assay was about 50 viral RNA molecules per milliliter of plasma. On the other hand, the threshold for the method of the invention at which the number of 2-LTR circles was conservatively estimated to give a positive result was set at 1 molecule or circle per million PBMC (roughly about 0.1 to 1 ml whole blood). Higher thresholds could be set, but such thresholds may lead to more false negatives. Considering the consequences of false negatives, the lowest practical threshold should be used. The results are summarized in Table 1.

TABLE 1

| | # Viral RNA/ | # 2-LTR Circles/ | Months without |
|---|---|---|---|
| Patient | ml Plasma | 10⁶ cells | Detectable Viral RNA |
| 1 | <50 | 20 | N/A |
| 2 | 155 | 25 | 8 |
| 3 | <50 | 47 | 12 |
| 4 | <50 | 872 | 9 |
| 5 | N/A | 13 | N/A |
| 6 | <50 | <1 | 9 |
| 7 | <50 | 6200 | 7 |
| 8 | <50 | <1 | 17 |
| 9 | N/A | 1 | N/A |
| 10 | <50 | 1 | N/A |
| 11 | <50 | 240 | 19 |
| 12 | 121 | 36 | 19 |
| 13 | N/A | 3 | 15 |
| 14 | <50 | 48 | 10 |
| 15 | <50 | <1 | 24 |
| 16 | <50 | 1 | 9 |
| 17 | <50 | 3 | 15 |
| 18 | <50 | <1 | 16 |
| 19 | <50 | 271 | 15 |
| 20 | 69 | 117 | 4 |

Table 1 illustrates the unexpectedly superior sensitivity of 2-LTR circle detection as compared to the standard plasma viral load assay. Some HIV-positive individuals, who do not have detectable plasma virus, nevertheless harbor newly HIV-infected blood cells, as indicated by the presence of 2-LTR circles (i.e., patients 1-5, 7, 9-14, 16, 17, 19, and 20). These individuals should not cease antiviral therapy, since infectious virus is still present in the body.

On the other hand, in some patients with undetectable plasma virus, no 2-LTR circles were detected in their PBMC (i.e., patients 6, 8, 15, and 18). These individuals may have completely eradicated HIV from their bodies, and are candidates for removal form antiviral therapy.

### Example 2

The stability of 2-LTR circle forms of viral DNA were initially examined in acutely infected cells in vitro, CD4⁺ MT-4 T cells and Jurkat-CCR5 cells were infected with the X4 variant HIV-1_{LAI} and the R5 variant HIV-1_{ADA}, respectively. Synthesis of viral cDNA was allowed to proceed for 24 hours, and further rounds of virus infection and cDNA synthesis were then restricted by the addition of reverse transciptase inhibitors ZDV (5 μM) or Nevirapine (1 μM) to HIV-1_{LAI} and HIV-1_{ADA} infected cells, respectively. Cells were then maintained in the presence of the RT inhibitors.

The experimental procedures used in Examples 2-4 are briefly described,

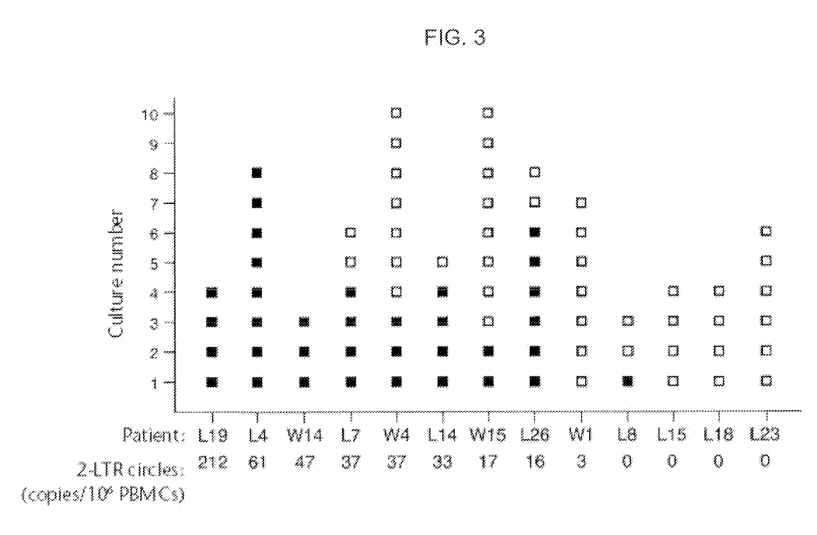

The relationship between 2-LTR circle frequency and either the duration of undetectable plasma viral RNA or the frequency of positive virus co-cultures was examined using Spearman's correlation coefficient. Mean frequency of positive co-cultures 2-LTR circle positive individuals and 2-LTR circle negative individuals as shown in FIG. 3 was further compared by a paired t-test.

Ficoll-purified PBMC (2-40×10⁺) were collected by centrifugation at 300×g for 2 minutes, Cell pellets were resuspended in buffer PI and extrachromosomal DNA was purified by QIAprep™ spin miniprep kit (Qiagen, Valencia, Calif.) using the modification for the isolation of low copy number plasmids as recommended by the manufacturer. Chromosomal DNA was recovered from the sodium acetate—SDS precipitate using DNAzol™ reagent (Life Technologies, Gaithersburg, Md.) according to the manufacturer's protocol. Total cellular DNA was purified us an Isoquick™ nucleic acid extraction kit (ORCA Research, Bothell, Wash.).

2-LTR circle Junctions were amplified from 10-30 μl of extrachromosomal DNA in a 50 μl reaction containing 1× HotStarTaq™ buffer, 200 nM dNTPs, 400 nM primers, and 1.5 units HotStarTaq™ (Qiagen, Valencia, Calif.). The reverse primer was 5′-cagatctggtctaaccagaga-3′ (SEQ ID NO: 1), and the forward primer was 5′-gtaactagagatccctcagac-3 (SEQ ID NO:2), which annealed to nucleotides 9157-9137 HIV-1 LTR region) and nucleotides 130-150 (HIV-1 LTR U5 region) of HIV-1_{LAI}, respectively (see GenBank Accession No. K02013 for numbering). After an initial denaturation step (95′C, 10 minutes), PCR amplification proceeded for 45 cycles (95° C., 30 seconds; 60° C., 30 seconds; 72° C., 60 seconds) followed by a final extension (72° C., 5 minutes).

To control for the effect of sequence polymorphisms at primer binding sites, amplification was performed with internal primers which were reversed in orientation to those listed above. Amplification with the internal LTR primers proceeded for 35 cycles using conditions outlined above. Polymorphisms in the region of the LTR that is recognized by the fluorogenic probe can affect annealing of the probe and potentially result in “false negatives.” Consequently. Taqman reaction products were subsequently analyzed on agarose-TBE gels and stained with ethidium bromide to ensure that those reactions did not contain episome-specific PCR products. For quantitation of 2-LTR circle frequency in patient PBMC. PCR reactions were performed using an ABI prism 7700 sequence detection system with the addition of 200 nM fluorogenic probe (5′-agtggcgagccctcagatgctge-3′; SEQ ID NO:3) to the reaction. The probe anneals to nucleotides 9081-9103 of HIV-1_{LAI} and was modified with 6-FAM (6-carboxyfluorescein) reporter dye on the 5′ end and 6-TAMRA (6-carboxytetramethylrhodamine) quencher dye on the 3′ end. Copy number estimates of 2-LTR circles were determined by extrapolation from is plot of standards versus band intensity or by using the ABI prism 7700 quantitation software. Per sequencing, 2-LTR circle junctions were cloned into a TA cloning vector (Invitrogen, San Diego, Calif.) and analyzed on an ABI 377 DNA sequencer according to the manufacturer's protocol.

Patient PBMC were separated by Ficoll-Paque (Amersham-Pharmacia) and depleted of CD8⁺ T lymphocytes using antibody-coated beads (DynaI). Cells were seeded in flasks in aliquots of 1×10⁷ cells in RPMI 1640 medium supplemented with 10% fetal calf serum and activated by PHA (5 μg/ml) for 12 hours. CD8⁺-depleted PBMC from seronegative individuals were activated for 12 hours with PHA and added in equal numbers to flasks of patient PBMC together with 20 IU/ml of interleukin-2 (Genzyme). At weekly intervals, half of the culture supernatant was replaced with fresh medium containing 20 IU/ml IL-2 and 10⁷ freshly isolated, CD8⁺-depleted, PHA activated donor PBMC from HIV-1 seronegative individuals. HIV-1 Gag p24 antigen in culture supernatants was evaluated by ELISA (Beckman Coulter) after 4 weeks.

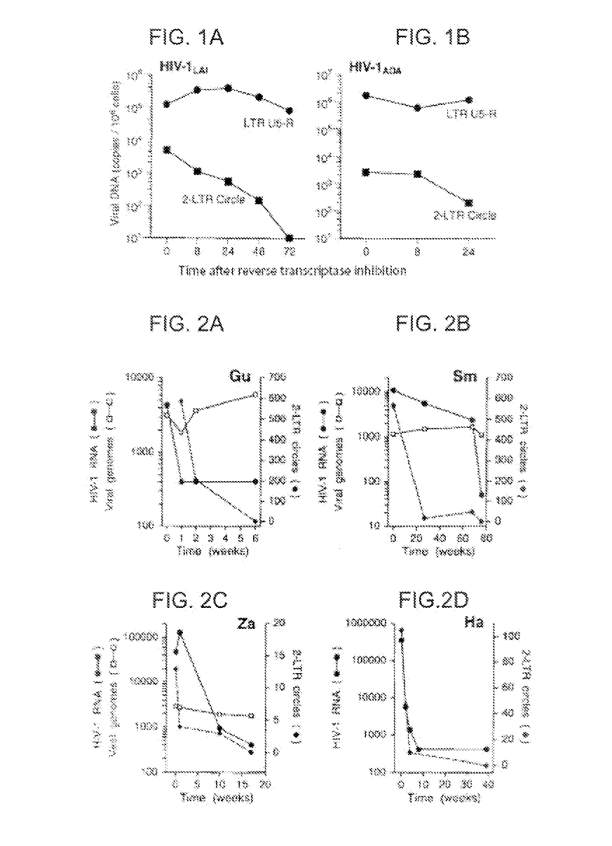

Within 24-48 hours following addition of the RT inhibitors, 2-LTR circle number fell by over ten fold in both HIV-1_{LAI} and HIV-1_{ADA} infected cells (FIGS. 1A and 1B). The copy number of other viral DNA forms identified by the internal LTR primers (predominantly linear and integrated viral genomes) remained relatively constant over the same interval. Thus, 2-LTR circles appeared to be labile intermediates in the virus lifecycle.

### Example 3

Whether 2-LTR circles were labile in vivo was next evaluated. PBMC samples were obtained from four HIV-1 infected individuals (Gu, Sm, Za, Ha) who, following adjustment of their antiretroviral regimens to more potent combinations, exhibited steady declines in plasma viral RNA levels. Patient Gu, who had been maintained on a two-drug RT inhibitor combination, was subsequently changed (week 0) to a three-drug regimen (ZDV/3TC/NFV). Patient Sm, who had been on a two-drug regimen (ZDV/3TC) was changed at week 68 to ddI/EFV/NFV. Patient Za, who had been on a four-chug regimen (3TC/D4T/ddI/NFV) was adjusted (week 1) to ZDV/ddC/NTV/RTV. Patient Ha, previously on a three-drug regimen (ZDV/ddI/NVP) was subsequently adjusted (week 0) to D4T/3TC/NVP. Marked declines in 2-LTR circle copy number were observed over the interval in which there was a rapid drop in levels of plasma viral RNA (FIGS. 2A-2D). In contrast, when samples were analyzed in parallel with internal LTR primers, HIV-1 viral genome levels (detected via cDNA) fluctuated by no more than three fold (FIGS. 2A-2C). Collectively, the results suggest that 2-LTR circles are labile, both in vitro and in vivo, relative to integrated viral genomes.

### Example 4

A larger patient population than that of Example 1 was then examined, the 2-LTR HIV-1 episomes were examined in 63 patients (four of whom were included in the study described in Example 1) who, through treatment with high activity antiretroviral therapy (HAART), had undetectable levels of plasma viral RNA for sustained periods of time (Table 2), Fifty of these patients (80%) had undetectable levels of plasma viral RNA (assay limit of sensitivity was 400 copies/ml) for 12 months or longer (Table 2). Of these 50 patients, 24 (48%) exhibited undetectable levels of plasma viral RNA for 12 months or more using an assay with a sensitivity of 250 copies/ml. In 48 of the 63 patients (76%), 2-LTR circles were detected in their PBMC (Table 2), 2-LTR circle copy numbers ranged from less than 1 copy/10⁶ PBMC to 620 copies/10⁶ PBMC. There did not appear to be any significant relationship between the frequency of 2-LTR circles in patient PBMC and the time during which plasma viral RNA was undetectable. Thin data indicated that labile replication intermediates are present in a substantial proportion of HIV-1 infected individuals who exhibit sustained suppression of plasma viral RNA while on HAART. 2-LTR circles were not detectable in PBMC from 15 (24%) patients (Table 2).

Table 2 below lists AIDS patients on HAART and the level of 2-LTR circles and viral RNA in the blood. The abbreviations for Table 2 are as follows. Anti-retroviral therapy: ZDV, Zidovudine; 3TC, Lamivudine; D4T, Stavudine; ddl, Didanosine; NVP, Nevirapine; RTV, Ritonavir; EFV, Efavirenz; SQV, Saquinavir; IDV, Indinavir; NFV, Nelfinavir; ddC, Zalcitabine; and ABV, Abacavir. CD4⁺ T cell measurements were determined at or just prior to time PBMC were collected for PCR analysis of viral cDNA intermediates.

For the column labeled “Period of Undetectable Viral RNA,” plasma viral RNA was detected using an assay with a sensitivity of about 400 copies/ml. Numbers in parentheses indicated the period for which viral RNA was below the level of detection using a second assay with a sensitivity of 50 copies/ml. Plasma viral RNA measurements were determined approximately every three months.

The 2-LTR circle copy number in most cases were determined in duplicate on independent PBMC samples. Values less than 1 indicated that more than 1 million PBMC were required for detection of 2-LTR circles.

The total number of PBMC from which extrachromosomal DNA was isolated and analyzed for the presence of 2-LTR circles was determined as follows. In all patients, 2-LTR circles were quantitated by fluorescence-based PCR using Taqman software (ABI Prism 7700 Software). Similar 2-LTR circle numbers were obtained when scruples were quantitated by comparison of PCR band intensity to a standard dilution of synthetic 2-LTR circles.

TABLE 2

| | | | Period of | | |
|---|---|---|---|---|---|
| | | CD4⁺ T | Undetectable | 2-LTR circles | #PBMC |
| Patient | | Cells | Viral RNA | (Copies/10⁶ | Analyzed |
| Number | Drug Regimens | (cells/ml) | (months) | PBMC) | (millions) |
| W1 | RTV, ZDV, 3TC | 475 | 23 (14) | 3 | 1.0 |
| W2 | NFV, ZDV, 3TC | 827 | 13 (13) | <1 | 5.5 |
| W3 | IDV, D4T, 3TC | 436 | 23 (14) | 27 | 1.0 |
| W4 | IDV, D4T, 3TC | 505 | 22 (12) | 37 | 1.0 |
| W6 | IDV, D4T, 3TC | 248 | 19 (11) | 15 | 1.0 |
| W7 | SQV, D4T, 3TC | 443 | 19 (13) | 8 | 1.0 |
| W8 | ddl, D4T | 870 | 18 (15) | <1 | 4.0 |
| W9 | NFV, D4T, 3TC | 641 | 22 (11) | 59 | 1.0 |
| W10 | IDV, ZDV, 3TC | 656 | 22 (15) | <1 | 4.0 |
| W11 | IDV, ZDV, 3TC | 344 | 22 (15) | 65 | 1.0 |
| W12 | ZDV, 3TC, DLV | 626 | 26 (16) | <1 | 5.5 |
| W13 | NFV, ZDV, 3TC | 699 | 13 (13) | <1 | 5.5 |
| W14 | NFV, D4T, 3TC | 685 | 21 (15) | 47 | 1.0 |
| W15 | NFV, STC, NVP | 866 | 25 (12) | 17 | 1.0 |
| W16 | RTV, D4T, 3TC | 572 | 22 (14) | 2 | 5.5 |
| W17 | IDV, ZDV, 3TC | 364 | 26 (15) | 31 | 1.0 |
| W18 | IDV, ZDV, 3TC | 119 | 21 (16) | <1 | 2.0 |
| W19 | SQV, ZDV, 3TC | 153 | 16 (10) | 4 | 4.0 |
| W20 | IDV, ZDV, 3TC | 360 | 27 (15) | <1 | 4.0 |
| W21 | NFV, D4T, 3TC | 208 | 13 (13) | <1 | 2.0 |
| W22 | D4T, 3TC | 495 | 23 (15) | <1 | 4.0 |
| W28 | NFV, ddl, D4T | 527 | 22 (8) | 9 | 1.0 |
| W30 | D4T, 3TC | 575 | 22 (17) | <1 | 4.0 |
| M1 | NFV, D4T, NVP | 287 | 14 (9) | 31 | 1.0 |
| M3 | IDV, ddl, NVP | 440 | 16 (7) | 22 | 1.0 |
| M4 | IDV, ZDV, 3TC | 586 | 13 (ND) | 264 | 1.0 |
| M6 | NFV, ZDV, 3TC | 317 | 24 (7) | 63 | 1.0 |
| M7 | NFV, 3TC, NVP | 175 | 11 (ND) | 4 | 5.5 |
| M8 | IDV, ZDV, 3TC, NVP | 357 | 13 (2) | 15 | 1.0 |
| M12 | NFV, D4T, 3TC | 749 | 12 (7) | 35 | 1.0 |
| M13 | ZDV, 3TC, EFV | 670 | 10 (0) | 67 | 1.0 |
| M14 | IDV, ZDV, 3TC | 728 | 14 (14) | 41 | 1.0 |
| M15 | IDV, ZDV, 3TC | 565 | 10 (10) | 82 | 1.0 |
| M16 | NFV, 3TC, NVP | 403 | 12 (8) | 3 | 4.0 |
| L2 | 3TC, D4T, RTV | 852 | 8 (8) | 5 | 1.0 |
| L3 | ZDV, 3TC, IDV | 448 | 12 (8) | 10 | 1.0 |
| L4 | ZDV, 3TC, RTV | 978 | 21 (12) | 180 | 1.0 |
| L6 | D4T, RTV, SQV | 577 | 10 (7) | <1 | 4.0 |
| L7 | D4T, ddl, NVP | 394 | 11 (7) | 610 | 1.0 |
| L8 | ZDV, 3TC, NFV | 173 | 17 (8) | <1 | 1.0 |
| L9 | 3TC, D4T, EFV | 482 | 8 (5) | <1 | 2.2 |
| L11 | ZDV, 3TC, RTV | 615 | 19 (12) | 84 | 1.0 |
| L12 | 3TC, D4T, RTV | 389 | 19 (6) | 7 | 1.0 |
| L13 | D4T, SQV, NFV | 312 | 15 (3) | <1 | 7.8 |
| L14 | 3TC, D4T, IDV | 375 | 14 (7) | 116 | 1.0 |
| L15 | 3TC, RTV, SQV, ABV | 91 | 30 (17) | <1 | 1.5 |
| L16 | 3TC, D4T, SQV, RTV | 575 | 12 (12) | 4 | 8.1 |
| L17 | 3TC, D4T, SQV | 198 | 15 (15) | 14 | 1.0 |
| L18 | ZDV, 3TC, IDV | 175 | 16 (13) | <1 | 10.2 |
| L19 | 3TC, D4T, RTV, SQV | 499 | 15 (6) | 620 | 1.0 |
| L22 | ZDV, D4T, IDV | 223 | 14 (12) | 6 | 1.0 |
| L23 | 3TC, ddC, IDV | 534 | 14 (12) | <1 | 4.8 |
| L26 | 3TC, D4T, SQV, NFV | 911 | 17 (6) | 36 | 1.0 |
| L27 | ZDV, 3TC, IDV | 185 | 17 (17) | <1 | 3.2 |
| L28 | D4T, ABV, EFV | 80 | 8 (8) | 275 | 1.0 |
| L29 | ZDV, ddC, SQV, NFV | 121 | 21 (1) | 3 | 2.0 |
| L32 | 3TC, D4T, EFV | 219 | 7 (1) | <1 | 10.0 |
| L33 | 3TC, D4T, IDV | 610 | 16 (1) | <1 | 14.4 |
| L36 | ddl, D4T, NFV | 172 | 14 (4) | 2 | 5.6 |
| L37 | ZDV, ddC, 3TC, IDV | 279 | 13 (7) | <1 | 5.6 |
| L41 | ZDV, 3TC, RTV | 990 | 22 (1) | 100 | 1.0 |
| L42 | 3TC, D4T, SQV | 117 | 18 (1) | <1 | 2.0 |
| L46 | 3TC, D4T, NFV | 180 | 7 (1) | 4 | 20.0 |

It was suspected that in 2-LTR circle positive patients, there would also be cells harboring replication competent virus. To investigate this, high-input viral co-culture assays were performed on PBMC from nine 2-LTR circle positive and four 2-LTR circle negative patients. The results are shown in FIG. 3. Replication competent virus could readily be isolated from eight of the nine patients who were 2-LTR circle positive. Virus could not be isolated from patient W1 who had a very low circle copy number. Intriguingly, infectious virus could not be isolated from three patients who were 2LTR circle negative even though co-culture was conducted on between 40 and 60 million CD8⁺ depleted patient PBMC. In patient L8 who was also 2LTR circle negative, only one of three cultures yielded infectious virus (FIG. 3), Collectively, these results suggested a correlation between the presence of 2-LTR circles and cells harboring replication competent virus. Plasma based viral RNA assays therefore, unlike the 2-LTR circle assay, failed to reveal the full extent of viral activity in infected individuals who are being treated with HAART.

This study has important implications for the development of strategies to eradicate virus replication in HIV-1 infected individuals. Although complete elimination of HIV-1 replication may be difficult with current antiretroviral regimens, this study suggests instances in which even the most sensitive assays fail to reveal ongoing replication in some well suppressed patients. It is also likely that, as more potent antiretrovirals enter the clinic, ongoing or “covert” virus replication may be arrested in a higher percentage of patients. A better understanding of the nature of the reservoir which sustains virus replication in aviremic patients on HART may lead to the development of more effective strategies tar arrest of virus replication. Monitoring of the 2-LTR, as a superior surrogate marker for viral replication, can be integral to the understanding of viral reservoirs.

### Example 5

2-LTR circle DNA is obtained from a subject, who is well-suppressed on HAART, and the sequence of the viral DNA encoding the V3 loop of the viral envelope glycoprotein, which is contained within 2LTR amplicons generated with specific primers, is determined. Tropism is predicted based on the deduced sequence of amino acids within the V3 loop of the viral envelope glycoprotein. The overall charge of amino acids within the VS loop strongly predicts the X4 versus R5 tropism of the envelope. A computer algorithm is used to predict tropism from the net charge of amino acids within the V3 loop. This is then optionally corroborated by inserting amplified V3 envelope sequences into an infectious molecular clone of HIV-1 Tropism of this recombinant virus is then assessed all cell lines that express either the CXCR4 co-receptor or the CCR5 co-receptor.

Based on the tropism determined by this method, an appropriate treatment is selected.

### Other Embodiments

It is to be understood that while the invention has been described in conjunction with the detailed description thereof, the foregoing description is intended to illustrate and not limit the scope of the invention, which is defined by the scope of the claims. Other aspects, advantages, and modifications are within the scope of the following claims.

1. A method of determining co-receptor tropism of replication competent virus in an HIV-positive subject who has less than 50 cell-free viral RNA molecules/ml of serum, the method comprising:
providing a sample comprising a cell from the subject;
detecting an HIV 2-LTR circle DNA molecule in the sample; and
determining the co-receptor tropism of the 2-LTR circle DNA;
wherein the co-receptor tropism of the 2-LTR circle DNA indicates the co-receptor tropism of the replication competent virus in the subject.

2. The method of claim 1, wherein determining the co-receptor tropism of the 2-LTR circle comprises determining the genotype of the DNA.

3. The method of claim 2, wherein the DNA molecule is amplified before determining the genotype of the DNA using polymerase chain reaction (PCR) with primers specific for an envelope protein.

4. The method of claim 3, wherein the envelope protein is gp120 or gp41.

5. The method of claim 1, wherein determining the co-receptor tropism of the 2-LTR circle comprises performing a phenotypic tropism assay.

6. The method of claim 1, wherein the subject is being treated with highly active antiretroviral therapy (HAART).

7. The method of claim 1, wherein the subject is a human.

8. The method of claim 1, wherein the cell is a peripheral blood mononuclear cell.

9. The method of claim 1, wherein cell-free HIV viral RNA cannot be detected in the blood of the mammal.

10. The method of claim 1, wherein the co-receptor tropism of the 2-LTR circle DNA indicates that the replication competent virus is primarily M-tropic.

11. The method of claim 1, wherein the co-receptor tropism of the 2-LTR circle DNA indicates that the replication competent virus is primarily T-tropic.

12. The method of claim 1, wherein the co-receptor tropism of the 2-LTR circle DNA indicates that the replication competent virus is primarily dual-tropic.

13. The method of claim 1, further comprising selecting an entry inhibitor based on the co-receptor tropism of the replication competent virus.

14. The method of claim 13, comprising selecting a CCR5-specific entry inhibitor based on the presence of co-receptor tropism for CCR5 or of dual tropism.

15. The method of claim 13, comprising selecting a CXCR4-specific entry inhibitor based on the presence of co-receptor tropism for CXCR4.

16. A method of treating an HIV-infected subject who has less than 50 cell-free viral RNA molecules/ml of serum, the method comprising:
providing a sample comprising a cell from the subject;
detecting an HIV 2-LTR circle DNA molecule in the sample;
determining the co-receptor tropism of the 2-LTR circle DNA;
determining co-receptor tropism of replication-competent virus in the subject based on the co-receptor tropism of the 2-LTR circle DNA;
selecting an entry inhibitor suitable for the HIV co-receptor tropism of the virus in the subject; and
administering an effective amount of the selected entry inhibitor to the subject, thereby treating the subject.

17. The method of claim 16, comprising selecting a CCR5-specific entry inhibitor for a subject in whom the co-receptor tropism is for a receptor other than CXCR4.

18. The method of claim 16, comprising selecting a CXCR4-specific entry inhibitor for a subject in whom the co-receptor tropism is for CXCR4.

19. The method of claim 1, further comprising confirming that the subject has less than about 50 cell-free viral RNA molecules/ml of serum.

20. The method of claim 18, further comprising confirming that the subject has less than about 50 cell-free viral RNA molecules/ml of serum.
